(19) United States
(12) Patent Application Publication
(10) Pub. No.: US 2010/0016423 A1
(43) Pub. Date: Jan. 21, 2010

(54) USE OF DRONEDARONE FOR THE TREATMENT OF PATIENTS WITH ARRHYTHMIA AND HAVING AN INCREASE OF CREATININE LEVEL DUE TO DRONEDARONE ADMINISTRATION

(75) Inventors: Sophie CLAUDEL, Montrouge (FR); Christophe GAUDIN, Saint Cloud (FR); Nacera HAMDANI, Colombes (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/431,830

(22) Filed: Apr. 29, 2009

Related U.S. Application Data
(60) Provisional application No. 61/048,732, filed on Apr. 29, 2008.

(30) Foreign Application Priority Data
Apr. 29, 2008 (EP) 08290407.9

(51) Int. Cl. A61K 31/343; A61P 9/06

(52) U.S. Cl. 514/469

(57) ABSTRACT

Methods of using dronedarone or a pharmaceutically acceptable salt thereof for the treatment of patients with arrhythmia, said patients having an increase of creatinine level due to dronedarone administration, said creatinine level increasing following dronedarone treatment initiation, said creatinine level increase reaching a plateau and being used as a new baseline, said creatinine level increase being reversible after dronedarone discontinuation.

The instant invention relates to a use of dronedarone or one of its pharmaceutically acceptable salts for preparing a medicament for the treatment of patients with arrhythmia, wherein said patients have an increase of creatinine level due to dronedarone administration and to a method of managing the risk due to an observation of serum creatinine increase leading to inappropriate management of patients with CHF (Congestive Heart Failure). The instant invention more specifically relates to a method of managing the risk due to an observation of serum creatinine increase in patients treated by dronedarone and by ACE inhibitors or angiotensin II antagonists or potassium sparing diuretics.

### BRIEF DESCRIPTION OF THE DRAWINGS

FIG. 1 displays Chart 1, which shows the relative risk (dronedarone 400 mg BID versus placebo) estimates with 95% confidence intervals (CI) according to baseline characteristics—Cardiovascular deaths—All randomized patients (EFC5555/ATHENA), wherein NYHA means New-York Heart Association (classification I to IV for cardiac insufficiency), LVEF means left ventricular ejection fraction, N indicates the number of patients, and

RR means relative risk.

FIG. 2 displays Chart 2, which shows the relative risk (dronedarone 400 mg BID versus placebo) estimates with 95% confidence intervals according to baseline characteristics—cardiovascular hospitalization—all randomized patients (EFC5555/ATHENA)

### DETAILED DESCRIPTION OF THE INVENTION

2-n-butyl-3-[4-(3-di-n-butylaminopropoxy)benzoyl]-5-methylsulfonamido-benzofuran, or dronedarone, and its pharmaceutically acceptable salts are described in the European patent EP 0 471 609 B1.

Dronedarone is an antiarrhythmic agent effective in the reduction of cardiovascular hospitalization and death in patients with atrial fibrillation or atrial flutter or with a history of atrial fibrillation or atrial flutter. By the oral route, dronedarone is administered at daily doses up to 800 mg, in one or several intakes.

Creatinine is an end-product of muscle metabolism, which is freely filtered at the glomerulus and not metabolized in the kidney. It is secreted by the proximal tubules by both the anionic and the cationic secretory pathways. Usually an increase in creatinine level is considered as a marker of decreased glomerular filtration and as a sign of renal impairment (Journal of Internal medicine, 1999, 246, 247-252).

During clinical trials with dronedarone, it has been observed that administration of this active principle is associated with a slight increase in the serum creatinine levels of the patient. This increase occurred early after treatment initiation (within two days). Creatinine levels remained stable during treatment and returned to baseline within three days after treatment discontinuation.

It has however been demonstrated that such an increase in the creatinine levels is not linked to renal impairment, but is due to decreased secretion of this substance at the kidney tubular level. In fact, in a specific study in healthy subjects, this increase was shown to be related to inhibition of creatinine secretion at the tubular level, with no effect on glomerular filtration or on renal blood flow. Hence dronedarone can not be considered as a nephrotoxic agent. This decrease in the tubular secretion of creatinine is also reported with other drugs such as cimetidine, trimethoprim or even amiodarone (Br. J. Clin. Pharmac., 1993, 36, 125-127).

This effect of dronedarone on the creatinine levels becomes of particular relevance when this agent is co-prescribed to patients receiving another therapy that could interact with kidney function. This is especially the case for the treatment of patients with cardiovascular disease such as patients with heart failure, coronary disease and other cardiovascular risk factors receiving ACE inhibitors and/or angiotensin II receptor antagonists and/or spironolactone or other potassium sparing diuretics. Indeed, such categories of active ingredients are known for their adverse effects on the renal function. In patients treated both by dronedarone and by ACE inhibitors and/or angiotensin II receptor antagonists and/or potassium sparing diuretics, an increase in creatinine levels might be misinterpreted by the physician as a sign of nephrotoxicity of ACE inhibitors or angiotensin II receptor antagonists or potassium sparing diuretics, leading to inappropriate discontinuation of these agents. These treatments have been shown to prevent mortality in patients with heart failure (Pfeffer M A et al., Effect of captopril on mortality and morbidity in patients with left ventricular dysfunction after myocardial infarction. Results of the survival and ventricular enlargement trial. The SAVE Investigators, N. Engl. J. Med. 1992 Sep. 3; 327(10):669-77; Pitt B et al., The effect of spironolactone on morbidity and mortality in patients with severe heart failure. Randomized Aldactone Evaluation Study Investigators, N. Engl. J. Med. 1999; 341:709-717; Pitt B et al., Eplerenone, a selective aldosterone blocker, in patients with left ventricular dysfunction after myocardial infarction., N. Engl. J. Med. 2003; 348:1309-1321) and in patients with coronary disease (Indications for ACE inhibitors in the early treatment of acute myocardial infarction: systematic overview of individual data from 100,000 patients in randomized trials. ACE Inhibitor Myocardial Infarction Collaborative Group. Circulation 1998; 97:2202-2212; Swedberg K et al., Effects of the early administration of enalapril on mortality in patients with acute myocardial infarction. Results of the Cooperative New Scandinavian Enalapril Survival Study II (CONSENSUS II)., N. Engl. J. Med. 1992; 327:678-684; Dickstein K et al., Effects of losartan and captopril on mortality and morbidity in high-risk patients after acute myocardial infarction: the OPTIMAAL randomised trial. Optimal Trial in Myocardial Infarction with Angiotensin II Antagonist Losartan, Lancet 2002; 360:752-760) or to prevent events, including cardiovascular mortality, in patients at cardiovascular risk (Yusuf S et al., Effects of an angiotensin-converting-enzyme inhibitor, ramipril, on cardiovascular events in high-risk patients. The Heart Outcomes Prevention Evaluation Study Investigators, N. Engl. J. Med. 2000 Jan. 20; 342(3):145-53). Inappropriate interruption of the treatment with ACE inhibitors or angiotensin II receptor antagonists or potassium sparing diuretics therefore exposes these patients to an increased cardiovascular morbidity and mortality.

The Applicant has now found a method for managing such a risk. The method according to the invention enables to decrease the risk of an inappropriate interruption of ACE inhibitors, of angiotensin II receptor antagonists treatment or of potassium sparing diuretics, which consequently enables to decrease the risk of morbidity and mortality of the patient.

The instant invention therefore relates to a method of managing the risk of an increased morbidity and mortality rate in patients treated by an association of dronedarone or one of its pharmaceutically acceptable salts and of at least a compound B selected from ACE inhibitors, angiotensin II receptor antagonists and potassium sparing diuretics. Said method is performed by performing the following steps:
a) initially measuring serum creatinine levels, such as within one week after initiation of treatment with dronedarone, which provides a reference level,
b) monitoring serum creatinine levels at regular intervals during treatment of the patient by the compound B (as recommended in the labelling of compound B),
c) if the serum creatinine levels increases above the reference level, clinical judgement should be made to determine if the increase is due to dronedarone, to compound B or to another cause,
d) to help clinical judgment and evaluate if the increase is due to dronedarone, dronedarone can be temporarily interrupted. The return to baseline within 5 days will be highly suggestive of dronedarone responsibility, as demonstrated in another clinical trial. If despite discontinuation, creatinine levels do not decrease, another cause including other treatments should be considered.
   If, by this mean, the increase is thought to be due to dronedarone, concomitant treatment with B can be pursued with regular monitoring of creatinine levels,
   if the increase in the serum creatinine level above the reference level is thought to be due to compound B, then labelling instructions of compound B should be followed and alternative treatment could be considered,
   if the increase in the serum creatinine level above the reference level is due to another cause than administration of dronedarone or of compound B, then appropriate treatment of such cause shall be undertaken,
e) repeating steps b), c) and d) for the duration of the treatment with dronedarone and compound B.

The instant invention further relates to a method of managing the risk of an increase in the mortality rate in patients treated by an association of dronedarone or one of its pharmaceutically acceptable salts and of at least a compound B selected from ACE inhibitors, angiotensin II receptor antagonists and potassium sparing diuretics, which method comprises the following steps:
a) initially measuring serum creatinine levels, which provides a reference level,
b) monitoring serum creatinine levels at regular intervals during treatment of the patient by dronedarone and by the compound B,
c) if the serum creatinine levels increases above the reference level, determining if the increase is due to dronedarone, to compound B or to another cause,
d) if the increase in the serum creatinine level above the reference level is due to dronedarone, then the treatment with dronedarone and compound B can be pursued; if the increase in the serum creatinine level above the reference level is due to compound B, then the treatment with compound B may be interrupted; if the increase in the serum creatinine level above the reference level is due to another cause than administration of dronedarone or of compound B, then appropriate treatment of such another cause shall be undertaken,
e) repeating steps b), c) and d) for the duration of the treatment with dronedarone and compound B.

The instant invention further relates to a method of treatment of patients with cardiac arrhythmia comprising the administration of dronedarone or one of its pharmaceutically acceptable salts to patients treated, in addition, by at least a compound B selected from ACE inhibitors, angiotensin II receptor antagonists and potassium sparing diuretics, which method comprises the following steps:
a) initially measuring serum creatinine levels, which provides a reference level,
b) monitoring serum creatinine levels at regular intervals during treatment of the patient by dronedarone and by the compound B,
c) if the serum creatinine levels increases above the reference level, determining if the increase is due to dronedarone, to compound B or to another cause,
d) if the increase in the serum creatinine level above the reference level is due to dronedarone, then the treatment with dronedarone and compound B can be pursued; if the increase in the serum creatinine level above the reference level is due to compound B, then the treatment with compound B may be interrupted; if the increase in the serum creatinine level above the reference level is due to another cause than administration of dronedarone or of compound B, then appropriate treatment of such another cause shall be undertaken,
e) repeating steps b), c) and d) for the duration of the treatment with dronedarone and compound B.

The instant method of treatment can be more specifically directed to the treatment of patients with atrial fibrillation or atrial flutter.

Examples of ACE inhibitors may be captopril, enalapril, perindopril, quinapril, lisinopril, ramipril, etc.

Examples of angiotensin II receptor antagonists may be losartan, valsartan, candesartan, telmisartan, irbesartan, etc.

Examples of potassium sparing diuretics may be spironolactone, eplerenone, etc.

Such compounds are usually prescribed for prevention and treatment of various pathologies of the cardiac function, such as cardiovascular disorders, congestive heart failure, left ventricular dysfunction or hypertension.

As regards to dronedarone, the daily dose by the oral route is up to 800 mg, in one or several intakes.

In specific cases, higher or lower dosages may be appropriate; these dosages are comprised within the scope of the present invention. According to usual practice, the dosage suitable to each patient is determined by the physician according to the administration route, the weight and response of the patient.

The appropriate unitary dosage forms for dronedarone comprise the oral forms, such as tablets, hard or soft gelatin capsules, powders, granules and oral solutions or suspensions, as well as the sublingual, buccal, intratracheal, intraocular, intranasal forms, the forms adapted to inhalation, topical, transdermal, sub-cutaneous, intramuscular or intra-venous delivery, the rectal forms and the implants. For the topical application, dronedarone may be used as creams, gels, ointments or lotions.

Concerning step a) of the methods of the invention, as mentioned before, in most patients with atrial fibrillation or atrial flutter the maximal increase in creatinine levels, compared to the level before administration of dronedarone, was smaller than 30%, with a mean increase ranging from 10 to 15% or more. Then during the treatment with dronedarone, mean creatinine levels remain stable, this corresponds to a plateau.

In step a) of the methods according to the invention, the serum creatinine levels may in particular be measured one week after the start of dronedarone administration to the patient.

By “initially” in step a), one may for instance mean “one week after initiation of treatment with dronedarone”, being understood that a few days less or more are also encompassed within the scope of the step a), so that the reference level for serum creatinine may be measured at 3 to 15 days, more particularly 3 to 11 days, for example, after start of dronedarone administration to the patient.

In step b) of the methods according to the invention, the monitoring of the serum creatinine levels at regular intervals means that the serum creatinine shall be measured over the total duration of the treatment with dronedarone and compound B, based on a schedule depending on the specific pathological state of the patient and on the prescription labelling for the compound B. Such monitoring may for example be performed every 2 months in patients with renal impairment.

Step c) of the methods according to the instant invention targets to identify whether dronedarone or compound B may be the cause of the increase in the creatinine level above the reference level, or if it may be due to another cause, i.e. an outside cause than the active principles administered to the patient. In fact, various pathological states can be responsible for transient or permanent creatinine elevation, for example diabetes or impaired cardiac function, deshydration, hypovolemia, renal impairment, drug toxicity etc. Thus, a deterioration of the pathological state of the patient under the treatment of dronedarone and of compound B may involve serum creatinine elevation.

Step c) may be achieved by temporarily interrupting treatment with dronedarone, while maintaining treatment with compound B. Indeed, compound B should not be primarily interrupted unless there is a specific reason, especially in CHF patients.

The temporary interruption of dronedarone treatment or dronedarone discontinuation may for example range from 1 to 2 weeks. Then the serum creatinine level shall be measured. If it returns to the reference level, then dronedarone treatment shall be reinstated. Indeed, this would mean that the increase in creatinine was due to dronedarone itself, and not related to any other concomitant disease including other drug toxicity.

On the other hand, if the creatinine level does not return to the reference level after the dronedarone treatment interruption, then it would imply that dronedarone is not the cause of the further creatinine increase. In such a case it shall be determined whether the increase is due to compound B, in which case treatment with compound B may be interrupted, or at least closely monitored to avoid renal impairment, or if the increase is due to another cause, as described above, in which case appropriate treatment of such other cause shall be undertaken. Appropriate treatment shall be appreciated by the physician depending on the patient's clinical condition and associated pathologies.

Another subject of the instant invention is the use of dronedarone for preparing a medicament for use in the treatment of arrhythmia, wherein dronedarone or one of its pharmaceutically acceptable salts is used in combination with a compound B selected from ACE inhibitors, angiotensin II receptor antagonists and potassium sparing diuretics and wherein said use involves the following steps:
a) initially measuring serum creatinine levels, which provides a reference level,
b) monitoring serum creatinine levels at regular intervals during treatment of the patient by dronedarone and by the compound B,
c) if the serum creatinine levels increases above the reference level, determining if the increase is due to dronedarone, to compound B or to another cause,
d) if the increase in the serum creatinine level above the reference level is due to dronedarone, then the treatment with dronedarone and compound B can be pursued; if the increase in the serum creatinine level above the reference level is due to compound B, then the treatment with compound B may be interrupted; if the increase in the serum creatinine level above the reference level is due to another cause than administration of dronedarone or of compound B, then appropriate treatment of such another cause shall be undertaken,
e) repeating steps b), c) and d) for the duration of the treatment with dronedarone and compound B.

In an embodiment, the subject of the instant invention is the use of, or an association of dronedarone or one of its pharmaceutically acceptable salts and at least a compound B selected from ACE inhibitors, angiotensin II receptor antagonists and potassium sparing diuretics for preparing a medicament for the treatment of patients with arrhythmia, said patients having an increase of creatinine level due to dronedarone administration, said patients being defined with the following steps:

a) initially measuring serum creatinine levels, which provides a reference level,
b) monitoring serum creatinine levels at regular intervals during treatment of the patient by dronedarone and by the compound B,
c₁) if the serum creatinine levels increases above the reference level, temporarily interrupting treatment with dronedarone, while maintaining treatment with compound B,
d₁) when the increase is reversible, treatment with said combination is pursued,
e₁) repeating steps b), c₁) and d₁) for the duration of the treatment with said combination.

In an embodiment, the subject of the instant invention is the use of, or an association of dronedarone or one of its pharmaceutically acceptable salts and at least a compound B selected from ACE inhibitors, angiotensin II receptor antagonists and potassium sparing diuretics for preparing a medicament for the treatment of patients with arrhythmia, said patients having an increase of creatinine level due to compound B administration, said patients being defined with the following steps:

a) initially measuring serum creatinine levels, which provides a reference level,
b) monitoring serum creatinine levels at regular intervals during treatment of the patient by dronedarone and by the compound B,
c₁) if the serum creatinine levels increases above the reference level, temporarily interrupting treatment with dronedarone, while maintaining treatment with compound B,
d₂) when the increase is not reversible, treatment with compound B may be interrupted,
e₂) repeating steps b), c₁) and d₂) for the duration of the treatment with said combination.

In an embodiment, the subject of the instant invention is the use of, or an association of dronedarone or one of its pharmaceutically acceptable salts and at least a compound B selected from ACE inhibitors, angiotensin II receptor antagonists and potassium sparing diuretics for preparing a medicament for the prevention of death of patients with cardiac arrhythmia, said patients having an increase of creatinine level due to dronedarone administration, said patients being defined with the following steps:

a) initially measuring serum creatinine levels, which provides a reference level,
b) monitoring serum creatinine levels at regular intervals during treatment of the patient by dronedarone and by the compound B,
c₁) if the serum creatinine levels increases above the reference level, temporarily interrupting treatment with dronedarone, while maintaining treatment with compound B
d₁) when the increase is reversible, treatment with said combination is pursued,
e₁) repeating steps b), c₁) and d₁) for the duration of the treatment with said combination.

In an embodiment, the subject of the instant invention is the use of, or an association of dronedarone or one of its pharmaceutically acceptable salts and at least a compound B selected from ACE inhibitors, angiotensin II receptor antagonists and potassium sparing diuretics for preparing a medicament for the prevention of death of patients with cardiac arrhythmia, said patients having an increase of creatinine level due to compound B administration, said patients being defined with the following steps:

a) initially measuring serum creatinine levels, which provides a reference level,
b) monitoring serum creatinine levels at regular intervals during treatment of the patient by dronedarone and by the compound B,
c₁) if the serum creatinine levels increases above the reference level, temporarily interrupting treatment with dronedarone, while maintaining treatment with compound B,
d₂) when the increase is not reversible, treatment with compound B may be interrupted,
e₂) repeating steps b), c₁) and d₂) for the duration of the treatment with said combination.
In an embodiment, the subject of the instant invention is the use of dronedarone or one of its pharmaceutically acceptable salts for preparing a medicament for the treatment of patients with arrhythmia, said patients having an increase of creatinine level due to dronedarone administration, said creatinine level increasing following dronedarone treatment initiation, said creatinine level increase reaching a plateau and being used as a new baseline, said creatinine level increase being reversible after dronedarone discontinuation.

In an embodiment, the subject of the instant invention is the use of dronedarone or one of its pharmaceutically acceptable salts for preparing a medicament for the treatment of patients with arrhythmia, said patients having an increase of creatinine level due to dronedarone administration, wherein said use involves the following steps:
i) initiating dronedarone treatment,
ii) measuring serum creatinine level increases following dronedarone treatment initiation, which provides a reference level, said serum creatinine level reaching a plateau,
iii) discontinuing temporarily dronedarone,
iv) measuring that the increase is reversible after dronedarone discontinuation.
   In an embodiment, the subject of the instant invention is the use of an association of dronedarone or one of its pharmaceutically acceptable salts and compound B as defined above for preparing a medicament for the treatment of patients with arrhythmia, said patients having an increase of creatinine level not due to compound B administration, said creatinine level increasing following dronedarone treatment initiation, said creatinine level increase reaching a plateau and being used as a new baseline, said creatinine level increase being reversible after dronedarone discontinuation.

In an embodiment, the subject of the instant invention is the use of an association of dronedarone or one of its pharmaceutically acceptable salts and compound B as defined above for preparing a medicament for the treatment of patients with arrhythmia, said patients having an increase of creatinine level not due to compound B administration, wherein said use involves the following steps:
i) initiating dronedarone treatment,
ii) measuring serum creatinine level increases following dronedarone treatment initiation, which provides a reference level, said serum creatinine level reaching a plateau,
iii) discontinuing temporarily dronedarone,
iv) measuring that the increase is reversible after dronedarone discontinuation.
   The creatinine level increase may be an increase by about 0.1 mg/dL.
   The above mentioned plateau may be reached after 3 to 15 days, more particularly 3 to 11 days, for example after seven days.
   The fact that the creatinine level increase is reversible may be measured within 5 days after dronedarone discontinuation.
   Mention may be made that the expressions “reference level” and “new baseline” are equivalent.
   Mention may be made that the plateau is defined by the way of the measurement of serum creatinine level.
   Mention may be made that serum creatinine level is measured from a blood sample, said blood sample being obtained from a patient.

In an embodiment, the subject of the instant invention is a method of treatment of patients with arrhythmia comprising

i) initiating administration of dronedarone or a pharmaceutically acceptable salt thereof in said patient;

ii) obtaining a blood sample from the patient following initiation of administration of dronedarone, or a pharmaceutically acceptable salt thereof, to the patient; and

iii) measuring the serum creatinine levels from the blood sample to provide a reference level. In some embodiments, the patient is also being treated with a compound selected from the group consisting of ACE inhibitors, angiotensin II receptor antagonists and potassium sparing diuretics. In some embodiments, the method is for the treatment of patients with atrial fibrillation or atrial flutter. In some embodiments, the serum creatinine level in step iii) is measured within 3 to 15 days, more particularly within 3 to 11 days after initiation of treatment with dronedarone. In some embodiments, the serum creatinine level in step iii) is measured within one week after initiation of treatment with dronedarone.

In an embodiment, the subject of the instant invention is a method of treatment of patients with arrhythmia comprising

i) initiating administration of dronedarone or a pharmaceutically acceptable salt thereof in said patient,

ii) obtaining a blood sample from the patient following initiation of administration to the patient of dronedarone, or a pharmaceutically acceptable salt thereof;

iii) measuring the serum creatinine level from the blood sample to provide a reference level;

iv) discontinuing administration of dronedarone, or a pharmaceutically acceptable salt thereof, temporarily;

v) obtaining a blood sample from the patient following discontinuance of administration of dronedarone or a pharmaceutically acceptable salt thereof to the patient; and

vi) measuring the serum creatinine level in the blood sample from the patient following discontinuance to provide a reference level. In some embodiments, the method is for the treatment of patients with atrial fibrillation or atrial flutter. In some embodiments, the patient is also being treated with a compound selected from the group consisting of ACE inhibitors, angiotensin II receptor antagonists and potassium sparing diuretics. In some embodiments, the serum creatinine level in step iii) is measured within 3 to 15 days, more particularly within 3 to 11 days after initiation of treatment with dronedarone. In some embodiments, the serum creatinine level in step iii) is measured within one week after initiation of treatment with dronedarone.

In an embodiment, the subject of the instant invention is a method of treatment of patients with arrhythmia, wherein dronedarone or one of its pharmaceutically acceptable salts is used in combination with at least a compound B selected from ACE inhibitors, angiotensin II receptor antagonists and potassium sparing diuretics, said method comprising

i) initiating administration of dronedarone, or a pharmaceutically acceptable salt thereof, in said patient;

ii) obtaining a blood sample from the patient following initiation of dronedarone, or a pharmaceutically acceptable salt thereof, administration to the patient,

iii) measuring the serum creatinine level in the blood sample to provide a reference level,

iv) obtaining a blood sample from the patient and measuring serum creatinine level during treatment of the patient by dronedarone, or a pharmaceutically acceptable salt thereof, and by the compound B,

v) determining the cause of an increase serum creatinine level above the reference level,

vi) continuing treatment of dronedarone and compound B when the increase is due to treatment with dronedarone or pharmaceutically acceptable salt thereof; or interrupting treatment with compound B when the increase is caused by treatment with compound B; or undertaking appropriate treatment when the increase is due to a cause other than administration of dronedarone or of compound B, and

vii) repeating steps iv) to vi) at regular intervals for the duration of the treatment with dronedarone and compound B. In some embodiments, the method is for the treatment of patients with atrial fibrillation or atrial flutter. In some embodiments, the serum creatinine level in step iii) is measured within 3 to 15 days, more particularly within 3 to 11 days after initiation of treatment with dronedarone. In some embodiments, the serum creatinine level in step iii) is measured within one week after initiation of treatment with dronedarone, or a pharmaceutically acceptable salt thereof.

In an embodiment, the subject of the instant invention is a method of providing dronedarone, or a pharmaceutically acceptable salt thereof, wherein said dronedarone or pharmaceutically acceptable salt thereof is provided along with information indicating that serum creatinine levels increase following treatment initiation of dronedarone, or a pharmaceutically acceptable salt thereof. In some embodiments the information comprises printed matter that indicates that serum creatinine levels increase following treatment initiation of dronedarone, or a pharmaceutically acceptable salt thereof. In some embodiments, the information comprises written material recommending that if an increase in creatininemia is observed, this value should be used as the new baseline. In some embodiments, the printed matter is a label.

The term “providing” includes selling, distributing, shipping, offering for sell, importing etc.

In an embodiment, the subject of the instant invention is a method of promoting the use of dronedarone or a pharmaceutically acceptable salt thereof, the method comprising the step of conveying to a recipient at least one message selected from the group consisting of:

(1) measure plasma creatinine values within 7 days after treatment initiation of dronedarone or a pharmaceutically acceptable salt thereof;
(2) if an increase in creatininemia is observed, this value should be used as the new baseline;
(3) serum creatinine level increase following treatment initiation of dronedarone or a pharmaceutically acceptable salt thereof;
(4) dronedarone, or a pharmaceutically acceptable salt thereof, causes an increase in serum creatinine;
(5) the elevation of serum creatinine level reaches a plateau; and
(6) an increase in serum creatinine level is reversible after discontinuation of dronedarone or pharmaceutically acceptable salt thereof.

In an embodiment, the subject of the instant invention is an article of manufacture comprising

a) a packaging material;

b) dronedarone or a pharmaceutically acceptable salt thereof; and

c) a label or package insert contained within the packaging material indicating that serum creatinine level may change following treatment initiation of dronedarone, or a pharmaceutically acceptable salt thereof.

In an embodiment, the subject of the instant invention is a package comprising dronedarone or a pharmaceutically acceptable salt thereof and a label, said label comprising a printed statement which informs a prospective user that an increase in serum creatinine has been observed following initiation of treatment with dronedarone or a pharmaceutically acceptable salt thereof.

In an embodiment, the subject of the instant invention is a package comprising dronedarone or a pharmaceutically acceptable salt thereof and a label, said label comprising a printed statement which recommends that if an increase in creatininemia is observed following treatment initiation of dronedarone, or a pharmaceutically acceptable salt thereof, the increased value should be used as a baseline.

In an embodiment, the subject of the instant invention is a method of transforming a patient treated by an association of dronedarone and at least one compound B selected from ACE inhibitors, angiotensin II receptor antagonists and potassium sparing diuretics by managing the risk of an increase in the mortality rate in said patient which method comprises
i) initiating dronedarone treatment,
ii) measuring serum creatinine level increases following dronedarone treatment initiation, which provides a reference level,
iii) measuring that the increase reaches a plateau,
iv) temporarily discontinuing dronedarone treatment, and
v) measuring that the increase is reversible after dronedarone discontinuation.
   A pharmaceutically acceptable salt of dronedarone may be hydrochloride salt.
   The uses described above as for example use of dronedarone or one of its pharmaceutically acceptable salts for preparing a medicament for the treatment of the above mentioned diseases may be understood as use of a compound or an association for the preparation of a medicament for the treatment of the above mentioned diseases or as use of a compound or an association for the treatment of the above mentioned diseases or as a method for the treatment of the above mentioned diseases or as a compound or an association for the prevention or the treatment of the above mentioned diseases.
   These compound and association may also be used for the prevention or the treatment of the above mentioned diseases.

The instant invention is illustrated by the clinical data below.

### Example 1

As a preliminary comment, it has been observed and later demonstrated in a clinical trial in 627 patients with a recent severe episode of Congestive Heart failure (ANDROMEDA) that among all prognostic factors, the most important risk factor for death in patients receiving the dronedarone concomitantly, was the absence of treatment with ACE inhibitors or angiotensin II receptor antagonists (table 1).

TABLE 1
Adjusted relative risk of time from randomization to death by prognostic
factors up to 16 Jan. 2003 - all randomized and treated patients with a
recent severe episode of CHF (EFC4966/ANDROMEDA)

| | | Adjusted relative risk (a) | | |
|---|---|---|---|---|
| | | Relative | | |
| Prognostic factor | Risk | risk | 95% CI (b) | P-value (c) |
| ACE inhibitor or | Intake/ | 0.21 | [0.097; 0.432] | 0.00003 |
| angiotensin II | No intake | | | |
| receptor antagonist | | | | |

(a) Determined from Cox regression model.
(b) Confidence Interval.
(c) Statistical significance.

### Example 2

Despite the equal distribution of treatment with ACE inhibitors/AII receptor antagonists at baseline, concomitant treatment with these drugs became unbalanced during the course of the ANDROMEDA study, as more patients in the dronedarone 400 mg BID group discontinued these treatments and fewer started them (table 2).

TABLE 2
Number (%) of patients according to distribution of ACE inhibitor/AII
receptor antagonist intake - all randomized and treated patients
with a recent severe episode of CHF (EFC4966/ANDROMEDA)

| | | Dronedarone |
|---|---|---|
| | Placebo | 400 mg BID |
| | (N = 317) | (N = 310) |
| Patients with ACE inhibitors or AII | 267 (84.2%) | 274 (88.4%) |
| receptor antagonists at baseline | | |
| Patients with ACE inhibitors or AII | 254 (80.1%) | 237 (76.5%) |
| receptor antagonists at baseline who | | |
| did not interrupt these treatments | | |
| Patients who started ACE inhibitors | 27 (8.5%) | 12 (3.9%) |
| or AII receptor antagonists after | | |
| baseline who did not interrupt these | | |
| treatments | | |
| Patients who discontinued treatment | 18 (5.7%) | 41 (13.2%) |
| with ACE inhibitors or AII receptor | | |
| antagonists | | |
| Patients who were never treated with | 18 (5.7%) | 20 (6.5%) |
| ACE inhibitors or AII receptor antagonists | | |

Among patients who interrupted, or did not start treatment with ACE inhibitors/AII receptor antagonists (hereinafter “ACE/AII”), more patients in the dronedarone 400 mg BID group died, as opposed to patients who never interrupted ACE inhibitors/AII receptor antagonists (3.6% and 4%, respectively in placebo and dronedarone 400 mg BID groups) as shown in table 3.

TABLE 3
Number (%) of patients who died according to the intake
of ACE inhibitors/AII receptors antagonists up to 16
Jan. 2003 - all randomized and treated patients with
a recent severe episode of CHF (EFC4966/ANDROMEDA)

| | | Dronedarone |
|---|---|---|
| | Placebo | 400 mg BID |
| | (N = 317) | (N = 310) |
| Never interrupted concomitant | 10/281 (3.6%) | 10/249 (4%) |
| ACE inhibitors or AII receptor | | |
| antagonists | | |
| Never took or interrupted | 2/36 (5.6%) | 15/61 (24.6%) |
| concomitant ACE inhibitors or | | |
| AII receptor antagonists | | |

### Example 3

Table 4 shows how an increase in creatinine level induces the physicians to interrupt the treatment with ACE/AII.

TABLE 4
Number (%) of patients who stopped ACE/AII and who had
increased serum creatinine during the period of treatment
- All randomized and treated patients with a recent
severe episode of CHF (EFC4966/ANDROMEDA)

| | | Dronedarone |
|---|---|---|
| | Placebo | 400 mg BID |
| Adverse effects | n = 317 | n = 310 |
| Blood creatinine increased | 0/18 (0.0%) | 13/41 (31.7%) |
| Renal impairment | 0/18 (0.0%) | 1/41 (2.4%) |

In the dronedarone 400 mg BID group, 14 of 41 patients who discontinued ACE/AII experienced “blood creatinine increased” or “renal impairment” adverse events, versus none among the 18 patients taking placebo in this category. This observation supports the hypothesis that an increase in serum creatinine observed in patients taking dronedarone may have led investigators to discontinue ACE/AII treatment (table 4).

### Example 4

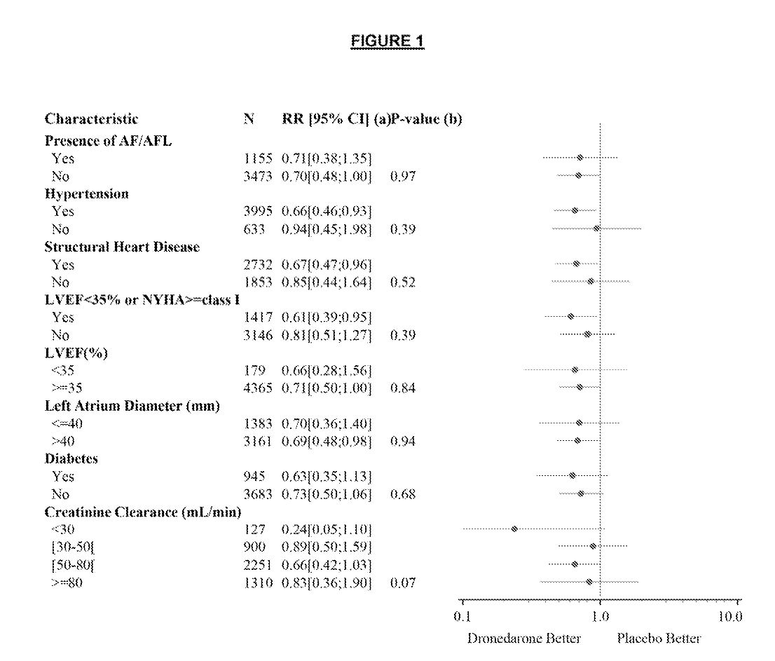

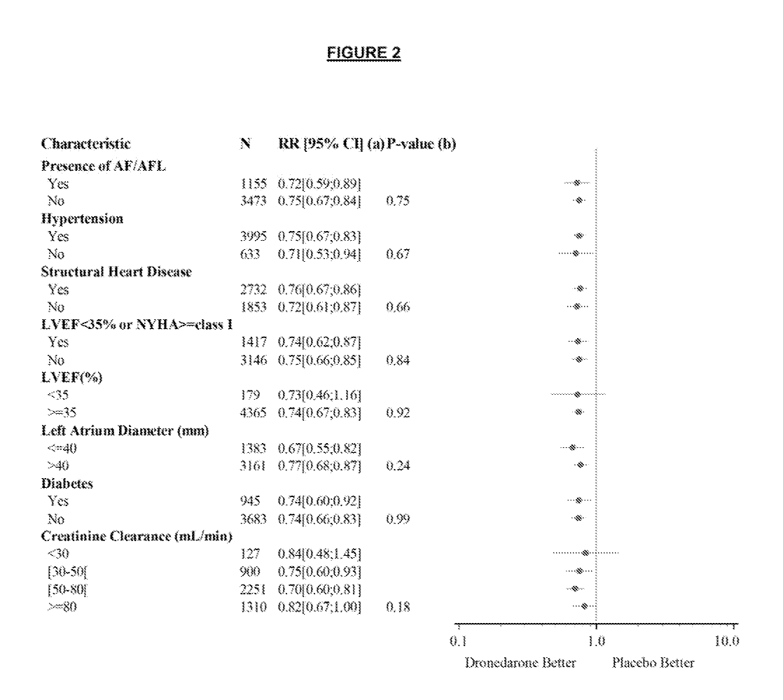

In the ATHENA trial, the above mentioned instructions to appropriately handle plasma creatinine increase and ACE inhibitors/AII receptor antagonists have been shown to be effective for the prevention of cardiovascular mortality and cardiovascular morbidity (hospitalizations for cardiovascular reasons), including in patients with different status of renal function, i.e. different values of creatinine clearance at baseline (chart 1 (FIG. 1) and chart 2 (FIG. 2)).

### Example 5

ACE/AII have been largely used in ATHENA clinical study and at the same rate in the dronedarone group and in the placebo group. More than 75% of the patients had received one of these compounds during the study. Table 5 shows the numbers and percentages of patients using various medications at the inclusion in the study, whereas table 6 shows the numbers and percentages of patients who received concomitant medications during the study. Diuretics with potassium sparing properties, and among them spirolonolactone, have also been prescribed in the ATHENA trial.

TABLE 5
Number (%) baseline selected medications - All randomized patients

| | | | Dronedarone | | | |
|---|---|---|---|---|---|---|
| | Placebo | | 400 mg BID | | Total | |
| | (N = 2327) | | (N = 2301) | | (N = 4628) | |
| Beta blocking agents (except sotalol) | 1641 | (70.5%) | 1628 | (70.8%) | 3269 | (70.6%) |
| ACE inhibitors or A II receptor antagonists | 1602 | (68.8%) | 1614 | (70.1%) | 3216 | (69.5%) |
| Oral anticoagulant | 1384 | (59.5%) | 1403 | (61.0%) | 2787 | (60.2%) |
| Diuretics | 1265 | (54.4%) | 1227 | (53.3%) | 2492 | (53.8%) |
| Diuretics other than spironolactone | 1224 | (52.6%) | 1187 | (51.6%) | 2411 | (52.1%) |
| Spironolactone | 136 | (5.8%) | 148 | (6.4%) | 284 | (6.1%) |
| Low dose of aspirin (<=365 mg) | 1019 | (43.8%) | 1018 | (44.2%) | 2037 | (44.0%) |
| Statins | 914 | (39.3%) | 878 | (38.2%) | 1792 | (38.7%) |
| Statins metabolized by CYP3A4 | 755 | (32.4%) | 737 | (32.0%) | 1492 | (32.2%) |
| Statins not metabolized by CYP3A4 | 166 | (7.1%) | 147 | (6.4%) | 313 | (6.8%) |
| Calcium antagonists with heart rate lowering | 307 | (13.2%) | 331 | (14.4%) | 638 | (13.8%) |
| effects | | | | | | |
| Digitalis | 308 | (13.2%) | 321 | (14.0%) | 629 | (13.6%) |
| Drugs interacting with the creatinine tubular | 237 | (10.2%) | 229 | (10.0%) | 466 | (10.1%) |
| secretion | | | | | | |
| Moderate inhibitors of CYP3A4 | 226 | (9.7%) | 214 | (9.3%) | 440 | (9.5%) |
| Other chronic antiplatelet therapy | 166 | (7.1%) | 126 | (5.5%) | 292 | (6.3%) |
| NSAID | 123 | (5.3%) | 114 | (5.0%) | 237 | (5.1%) |

TABLE 6
Number (%) of patients who received concomitant
medications - All randomized patients

| | | | Dronedarone | |
|---|---|---|---|---|
| | Placebo | | 400 mg BID | |
| | (N = 2327) | | (N = 2301) | |
| Beta blocking agents (except Sotalol) | 1860 | (79.9%) | 1785 | (77.6%) |
| ACE inhibitors/A II receptor antagonists | 1800 | (77.4%) | 1771 | (77.0%) |
| Oral anticoagulant | 1643 | (70.6%) | 1601 | (69.6%) |
| Diuretics | 1559 | (67.0%) | 1524 | (66.2%) |
| Diuretics other than Spironolactone | 1522 | (65.4%) | 1492 | (64.8%) |
| Spironolactone | 262 | (11.3%) | 257 | (11.2%) |
| Low dose of aspirin (<=365 mg) | 1231 | (52.9%) | 1225 | (53.2%) |
| Statins | 1131 | (48.6%) | 1044 | (45.4%) |
| Metabolized by CYP3A4 | 973 | (41.8%) | 909 | (39.5%) |
| Not metabolized by CYP3A4 | 305 | (13.1%) | 264 | (11.5%) |
| Digitalis | 574 | (24.7%) | 468 | (20.3%) |
| Calcium antagonists with heart rate | 490 | (21.1%) | 459 | (19.9%) |
| lowering effects | | | | |
| Drugs interacting with the creatinine | 434 | (18.7%) | 382 | (16.6%) |
| tubular secretion | | | | |
| Moderate inhibitors of CYP3A4 | 369 | (15.9%) | 323 | (14.0%) |
| NSAID | 359 | (15.4%) | 308 | (13.4%) |
| Other chronic antiplatelet therapy | 260 | (11.2%) | 182 | (7.9%) |

What is claimed is:

1. A method of treating arrhythmia in a patient comprising administering to said patient dronedarone or a pharmaceutically acceptable salt thereof, wherein said patient has an increase of creatinine level due to dronedarone administration, said creatinine level increasing following dronedarone treatment initiation, said creatinine level increase reaching a plateau and being used as a new baseline, said creatinine level increase being reversible after dronedarone discontinuation.

2. A method of treating arrhythmia in a patient comprising:
i) initiating treatment of dronedarone, or a pharmaceutically acceptable salt thereof, to said patient,
ii) measuring serum creatinine level increases following dronedarone treatment initiation, which provides a reference level, said serum creatinine level reaching a plateau,
iii) discontinuing temporarily dronedarone, or the pharmaceutically acceptable salt thereof, and
iv) measuring that the increase is reversible after discontinuation of dronedarone or the pharmaceutically acceptable salt thereof.

3. A method of treating arrhythmia in a patient comprising administering to said patient dronedarone or a pharmaceutically acceptable salt thereof, wherein, after said administration, said patient has an increase of creatinine level not due to administration of a compound B selected from ACE inhibitors, angiotensin II receptor antagonists and potassium sparing diuretics administration, which method comprises steps i) to iv) as defined in claim 2.

4. A method of managing the risk of an increase in the mortality rate in a patient treated by an association of dronedarone or a pharmaceutically acceptable salt thereof, and of at least a compound B selected from ACE inhibitors, angiotensin II receptor antagonists and potassium sparing diuretics which method comprises the steps i) to iv) as defined in claim 2.

5. A method of treating arrhythmia comprising administering dronedarone or a pharmaceutically acceptable salt thereof in combination with at least a compound B selected from ACE inhibitors, angiotensin II receptor antagonists and potassium sparing diuretics, and further comprising the following steps:
a) initially measuring serum creatinine levels, which provides a reference level,
b) monitoring serum creatinine levels at regular intervals during treatment of the patient by dronedarone or the pharmaceutically acceptable salt thereof, and by the compound B,
c) if the serum creatinine levels increases above the reference level, determining if the increase is due to dronedarone or the pharmaceutically acceptable salt thereof, to compound B or to another cause,
d) if the increase in the serum creatinine level above the reference level is due to dronedarone or the pharmaceutically acceptable salt thereof, then the treatment with dronedarone or the pharmaceutically acceptable salt thereof and compound B can be pursued; if the increase in the serum creatinine level above the reference level is due to compound B, then the treatment with compound B may be interrupted; if the increase in the serum creatinine level above the reference level is due to another cause than administration of dronedarone or the pharmaceutically acceptable salt thereof or of compound B, then appropriate treatment of such another cause shall be undertaken,
e) repeating steps b), c) and d) for the duration of the treatment with dronedarone or the pharmaceutically acceptable salt thereof and compound B.

6. The method according to claim 5 wherein said patient has an increase of creatinine level due to administration of dronedarone or the pharmaceutically acceptable salt thereof, said patient being defined with the following steps:
a) initially measuring serum creatinine levels, which provides a reference level,
b) monitoring serum creatinine levels at regular intervals during treatment of the patient by dronedarone or the pharmaceutically acceptable salt thereof and by the compound B,
c₁) if the serum creatinine levels increases above the reference level, temporarily interrupting treatment with dronedarone or the pharmaceutically acceptable salt thereof, while maintaining treatment with compound B,
d₁) when the increase is reversible, treatment with said combination is pursued,
e₁) repeating steps b), c₁) and d₁) for the duration of the treatment with said combination.

7. The method according to claim 5 wherein said patient has an increase of creatinine level due to compound B administration, said patient being defined with the following steps:
a) initially measuring serum creatinine levels, which provides a reference level,
b) monitoring serum creatinine levels at regular intervals during treatment of the patient by dronedarone or the pharmaceutically acceptable salt thereof and by the compound B,
c₁) if the serum creatinine levels increases above the reference level, temporarily interrupting treatment with dronedarone or the pharmaceutically acceptable salt thereof, while maintaining treatment with compound B,
d₂) when the increase is not reversible, treatment with compound B may be interrupted,
e₂) repeating steps b), c₁) and d₂) for the duration of the treatment with said combination.

8. A method for preventing death in a patient being treated with dronedarone, or a pharmaceutically acceptable salt thereof, and a compound B selected from ACE inhibitors, angiotensin II receptor antagonists and potassium sparing diuretics, wherein said patient has an increase of creatinine level due to administration of dronedarone or the pharmaceutically acceptable salt thereof, said method comprising:
a) initially measuring serum creatinine levels, which provides a reference level,
b) monitoring serum creatinine levels at regular intervals during treatment of the patient by dronedarone or the pharmaceutically acceptable salt thereof and by the compound B,
c₁) if the serum creatinine levels increases above the reference level, temporarily interrupting treatment with dronedarone or the pharmaceutically acceptable salt thereof, while maintaining treatment with compound B
d₁) when the increase is reversible, treatment with said combination is pursued,
e₁) repeating steps b), c₁) and d₁) for the duration of the treatment with said combination.

9. A method for preventing death in a patient being treated with dronedarone or a pharmaceutically acceptable salt thereof and a compound B selected from ACE inhibitors, angiotensin II receptor antagonists and potassium sparing diuretics, wherein said patient has an increase of creatinine level due to compound B administration, said method comprising:
a) initially measuring serum creatinine levels, which provides a reference level,
b) monitoring serum creatinine levels at regular intervals during treatment of the patient by dronedarone or the pharmaceutically acceptable salt thereof and by the compound B,
c₁) if the serum creatinine levels increases above the reference level, temporarily interrupting treatment with dronedarone or the pharmaceutically acceptable salt thereof, while maintaining treatment with compound B,
d₂) when the increase is not reversible, treatment with compound B may be interrupted,
e₂) repeating steps b), c₁) and d₂) for the duration of the treatment with said combination.

10. The method according to any one of claims 1 to 9, wherein said patient is being treated for atrial fibrillation or atrial flutter.

11. The method according to any one of claims 5 to 9, wherein the serum creatinine level in step a) is measured within 3 to 15 days after initiation of treatment with dronedarone or a pharmaceutically acceptable salt thereof.

12. The method according to any one of claims 5 to 9, wherein the serum creatinine level in step a) is measured within one week after initiation of treatment with dronedarone.

13. The method according to any one of claims 5 to 9, wherein the monitoring of the serum creatinine level is effected according to the prescription labelling for the compound B.

14. The method according to claim 5, wherein step c) is achieved by temporarily interrupting treatment with dronedarone, or the pharmaceutically acceptable salt thereof, while maintaining treatment with compound B.

15. The method according to claim 14, wherein the treatment interruption of dronedarone or the pharmaceutically acceptable salt thereof ranges from 1 to 2 weeks.

16. A method of managing the risk of an increase in the mortality rate in a patient treated by an association of dronedarone, or a pharmaceutically acceptable salt thereof, and of at least a compound B selected from ACE inhibitors, angiotensin II receptor antagonists and potassium sparing diuretics which method comprises the steps a) to e) as defined in claim 5.

17. A method of treating patients with cardiac arrhythmia comprising the administration of dronedarone to patients treated, in addition, by at least a compound B selected from ACE inhibitors, angiotensin II receptor antagonists and potassium sparing diuretics, which method comprising the steps a) to e) as defined in claim 5.

18. The method according to claim 16, for the treatment of patients with atrial fibrillation or atrial flutter.

19. A method of treating patients with arrhythmia comprising:
i) initiating administration of dronedarone or a pharmaceutically acceptable salt thereof in said patient;
ii) obtaining a blood sample from the patient following initiation of administration of dronedarone, or the pharmaceutically acceptable salt thereof, to the patient, and
iii) measuring the serum creatinine level from the blood sample to provide a reference level.

20. The method according to claim 18 further comprising:
iv) discontinuing administration of dronedarone, or the pharmaceutically acceptable salt thereof, temporarily, and
v) obtaining a blood sample from the patient following discontinuance of administration of dronedarone or the pharmaceutically acceptable salt thereof to the patient, and
vi) measuring the serum creatinine level in the blood sample from the patient following discontinuance.

21. The method according to claim 19 or claim 20, wherein the patient is also being treated with a compound selected from the group consisting of ACE inhibitors, angiotensin II receptor antagonists and potassium sparing diuretics.

22. A method of treating patients with arrhythmia, wherein dronedarone or a pharmaceutically acceptable salt thereof is administered in combination with at least a compound B selected from ACE inhibitors, angiotensin II receptor antagonists and potassium sparing diuretics, said method comprising
i) initiating administration of dronedarone, or the pharmaceutically acceptable salt thereof, in said patient,
ii) obtaining a blood sample from the patient following initiation of administration of dronedarone, or the pharmaceutically acceptable salt thereof, to the patient,
iii) measuring the serum creatinine levels in the blood sample to provide a reference level,
iv) obtaining a blood sample from the patient and measuring serum creatinine levels during treatment of the patient by dronedarone, or the pharmaceutically acceptable salt thereof, and by the compound B,
v) determining the cause of an increase serum creatinine levels above the reference level,
vi) continuing treatment of dronedarone or the pharmaceutically acceptable salt thereof and compound B when the increase is due to treatment with dronedarone or the pharmaceutically acceptable salt thereof; or interrupting treatment with compound B when the increase is caused by treatment with compound B; or undertaking appropriate treatment when the increase is due to a cause other than administration of dronedarone or the pharmaceutically acceptable salt thereof or of compound B, and
vii) repeating steps iv) to vii) at regular intervals for the duration of the treatment with dronedarone, or the pharmaceutically acceptable salt thereof, and compound B.

23. The method according to claim 22 for the treatment of patients with atrial fibrillation or atrial flutter.

24. The method according to claim 22, wherein the serum creatinine level in step iii) is measured within 3 to 15 days after initiation of treatment with dronedarone or the pharmaceutically acceptable salt thereof.

25. The method according to claim 22 or claim 23, wherein the serum creatinine level in step iii) is measured within one week after initiation of treatment with dronedarone or the pharmaceutically acceptable salt thereof.

26. A method of providing dronedarone, or a pharmaceutically acceptable salt thereof, comprising providing dronedarone or the pharmaceutically acceptable salt thereof along with information indicating that serum creatinine levels increase following treatment initiation of dronedarone or the pharmaceutically acceptable salt thereof.

27. The method according to claim 26 wherein the information comprises printed matter that indicates that serum creatinine level increases following treatment initiation of dronedarone or the pharmaceutically acceptable salt thereof.

28. The method according to claim 26 or claim 27 wherein the information comprises written material recommending that if an increase in creatininemia is observed, this value should be used as the new baseline.

29. The method according to claim 27 wherein the printed matter is a label.

30. The method according to claim 28 wherein the printed matter is a label.

31. A method of promoting the use of dronedarone or a pharmaceutically acceptable salt thereof, the method comprising the step of conveying to a recipient at least one message selected from the group consisting of:
(1) measure plasma creatinine values 7 days after treatment initiation of dronedarone or a pharmaceutically acceptable salt thereof;
(2) if an increase in creatininemia is observed, this value should be used as the new baseline;
(3) serum creatinine levels increase following treatment initiation of dronedarone or a pharmaceutically acceptable salt thereof;
(4) dronedarone, or a pharmaceutically acceptable salt thereof, causes an increase in serum creatinine;
(5) the elevation of serum creatinine level reaches a plateau; and
(6) an increase in serum creatinine levels is reversible after discontinuation of dronedarone or a pharmaceutically acceptable salt thereof.

32. An article of manufacture comprising
a) a packaging material;
b) dronedarone or a pharmaceutically acceptable salt thereof; and
c) a label or package insert contained within the packaging material indicating that serum creatinine levels may change following treatment initiation of dronedarone, or a pharmaceutically acceptable salt thereof.

33. A package comprising dronedarone or a pharmaceutically acceptable salt thereof and a label, said label comprising a printed statement which informs a prospective user that an increase in serum creatinine has been observed following initiation of treatment with dronedarone or a pharmaceutically acceptable salt thereof.

34. A package comprising dronedarone or a pharmaceutically acceptable salt thereof and a label, said label comprising a printed statement which recommends that if an increase in creatininemia is observed following treatment initiation of dronedarone, or a pharmaceutically acceptable salt thereof, the increased value should be used as a baseline.

35. A method of transforming a patient treated by an association of dronedarone and at least one compound B selected from ACE inhibitors, angiotensin II receptor antagonists and potassium sparing diuretics by managing the risk of an increase in the mortality rate in said patient which method comprises:
i) initiating treatment of dronedarone, or a pharmaceutically acceptable salt thereof, to said patient,
ii) measuring serum creatinine levels increase following treatment initiation of dronedarone or a pharmaceutically acceptable salt thereof, which provides a reference level, said serum creatinine level reaching a plateau,
iii) temporarily discontinuing treatment with dronedarone or a pharmaceutically acceptable salt thereof, and
iv) measuring that the increase is reversible after discontinuation of dronedarone or a pharmaceutically acceptable salt thereof.
